(19) United States
(12) Patent Application Publication
(10) Pub. No.: US 2014/0303242 A1
(43) Pub. Date: Oct. 9, 2014

(54) Sparstolonin B Based Pharmaceutical Agent to Treat Pathological Angiogensis

(71) Applicant: University of South Carolina, Columbia, SC (US)

(72) Inventors: Daping Fan, Columbia, SC (US); Susan Lessner, Newberry, SC (US); Henry Bateman, Columbia, SC (US)

(73) Assignee: University of South Carolina, Columbia, SC (US)

(21) Appl. No.: 14/056,336

(22) Filed: Oct. 17, 2013

Related U.S. Application Data
(60) Provisional application No. 61/809,614, filed on Apr. 8, 2013.

(51) Int. Cl. A61K 31/366; A61K 45/06

(52) U.S. Cl. CPC A61K 31/366; A61K 45/06
USPC 514/453; 435/375

(57) ABSTRACT

Anti-angiogenesis methods and compositions are described. The anti-angiogenic effect is provided by application of sparstolonin B or a derivative thereof to endothelial cells. The sparstolonin B or derivative thereof can inhibit angiogenesis by targeting endothelial cell cycle progression, cell migration, and chemotaxis.

### CROSS REFERENCE TO RELATED APPLICATION

This application claims filing benefit of U.S. Provisional Patent Application Ser. No. 61/795,404 having a filing date of Oct. 17, 2012 and U.S. Provisional Patent Application Ser. No. 61/809,614 having a filing date of Apr. 8, 2013, both of which are incorporated herein by reference.

### GOVERNMENT SUPPORT CLAUSE

This invention was made with government support under P20RR021949, P20RR016461, and P20RR021949 awarded by the National Institutes of Health. The government has certain rights in the invention.

### BACKGROUND

Sparstolonin B (SsnB) is a novel bioreactive compound isolated from Sparganium stoloniferum, an herb historically used in Traditional Chinese Medicine as an anti-tumor agent. SsnB has demonstrated anti-inflammatory properties, inhibiting Toll-like receptor mediated inflammation in isolated macrophages and in mice.

Angiogenesis refers to capillary formation from existing blood vessels. Angiogenesis occurs in several stages and involves interactions between cells, soluble factors, and extracellular matrix molecules. First, endothelial cells with the help of proteolytic enzymes, including matrix metalloproteases (MMPs) and the plasminogen activator system (PA), break down the basement membrane of an existing blood vessel and invade the surrounding tissues. The PA system is comprised of the serine proteases urokinase-type plasminogen activator (uPA) and tissue-type plasminogen activator (tPA) that convert plasminogen into its active form, plasmin. Plasmin degrades numerous extracellular matrix proteins, including fibronectin, laminin, and fibrin. Plasmin also helps activate many MMPs. After the basement membrane of the blood vessel is broken down, endothelial cells migrate into the surrounding tissue and proliferate. Growth factors and other soluble proteins in the ECM often facilitate and regulate this process. The growth factors that are stored bound to the ECM can be released as the ECM is degraded by proteases. Certain growth factors, such as vascular endothelial growth factor, act as chemoattractants that facilitate the migration of endothelial cells to certain locations. After migration and proliferation, the endothelial cells form a new lumen and start to secrete extracellular matrix molecules, ultimately forming a new capillary.

Angiogenesis plays an important role in many normal events in the body, including wound healing, embryogenesis, and female reproductive processes. During these normal processes, angiogenesis is highly regulated. Unregulated angiogenesis contributes to many pathological processes, such as rheumatoid arthritis, psoriasis, diabetic retinopathy, and tumor growth and metastasis. Clearly, angiogenesis is an important event in the human body.

Angiogenesis also contributes to atherosclerosis, which is a serious disease affecting a significant portion of the U.S. population and causing many deaths from conditions such as stroke and myocardial ischemia. There are many risk factors for atherosclerosis including hypertension, smoking, diabetes, high-fat diet, and dyslipidemia. Atherosclerosis specifically involves the formation of plaques within blood vessels, progressing through several stages. In the earliest stage, fatty streaks or atheromas form as lipids and lipoproteins deposit in the tunica intima of the blood vessel. A high ratio of LDL:HDL favors this event. The lipid deposits become oxidized and induce injury to the endothelial cells lining the blood vessel. Endothelial injury is a key step in atherosclerosis; endothelial cells may also be injured by denuding events or by more subtle hemodynamic changes. Cell adhesion molecules are upregulated in the endothelium, and inflammatory cells are attracted to the site of injury, including monocytes which differentiate into macrophages. As macrophages migrate to the area of oxidized lipids, scavenger receptors and toll-like receptors bind the lipids and help activate the macrophages to release inflammatory cytokines and mediators. The macrophages transform into foam cells as they take up oxidized lipids. Additional cells migrate to the area, including mast cells and leukocytes. An advanced lesion forms as smooth muscle cells from the tunica media proliferate and migrate to the plaque and form a fibrous cap. The smooth muscle cells may also transform into foam cells. An area of dead cells and cholesterol crystals forms within the plaque. A capillary network also grows within the plaque to help provide nutrients, gases, and inflammatory cells to the growing plaque. Blood vessels within the tunica adventitia, known as the vasa vasorum, supply this capillary network. As the plaque progresses, the fibrous cap weakens and ruptures, exposing the highly thrombogenic necrotic core region. Thrombus formation subsequently ensues. The thrombus may occlude the vessel or become an embolus that travels to another region of the body.

Atherosclerotic plaque rupture is the leading cause of acute cardiovascular events such as myocardial infarction (“heart attack”) and stroke. In this process, the fibrous cap overlying an atherosclerotic plaque fails, resulting in rapid thrombus formation which may completely occlude the vessel, leading to ischemia and tissue death downstream of the blockage. Newly developed and recurring myocardial infarction afflicts approximately 1.1 million people in the USA per year, with a 40% fatality rate; 220,000 of these deaths occur without hospitalization. Roughly 75% of these clinical events are caused by atherosclerotic plaque rupture. Clearly, methods of stabilizing atherosclerotic plaques to prevent plaque rupture would have a significant clinical impact.

Research has shown a link between capillary networks within atherosclerotic plaques and plaque progression and rupture. Under normal physiological conditions, only thick-walled blood vessels such as the descending aorta and the common carotid artery contain an intramural capillary network. This network of blood vessels, known as the vasa vasorum, is situated in the tunica adventitia and outer third of the tunica media. It supplies the blood vessel wall with nutrients and allows gas exchange. Human atherosclerotic arteries have shown extensive intraplaque angiogenesis, often linked to the vasa vasorum. It has been suggested that hypoxia and/or reactive oxygen species are responsible for initiating new capillary formation. Capillary networks within atherosclerotic plaques serve as a source of inflammatory cells, nutrients, and mediators, allowing the neo-intima to expand beyond the normal wall thickness. Intraplaque angiogenesis is correlated with decreased plaque stability and increased incidence of plaque rupture, subsequent thrombus formation, and intra-arterial occlusion. Recent research has suggested that inhibiting angiogenesis may improve plaque stability and decrease the chances for plaque rupture. In addition, inhibiting angiogenesis may decrease plaque formation by limiting this supply of necessary factors to the plaque region.

Angiogenesis may be inhibited at multiple steps. Growth factors, including Vascular Endothelial Growth Factor (VEGF) and basic Fibroblast Growth Factor, are suitable targets. Current approaches to sequester VEGF include the use of soluble VEGF receptors and neutralizing antibodies against VEGF. Intracellular signaling molecules, including tyrosine kinases, can be inhibited to prevent receptor-mediated activation of endothelial cells. There also exist natural angiogenesis inhibitors, including endostatin, retinoids, and fibronectin fragment, which may be targeted for angiogenesis inhibition. Angiogenesis inhibitors often target endothelial cells through the inhibition of cell proliferation, migration, and protease production. Current research into angiogenesis inhibition has demonstrated ways to limit atherosclerosis. By inhibiting intraplaque angiogenesis, necessary factors, such as inflammatory cells, oxygen, and mediators, are not delivered to the growing plaque, diminishing plaque growth. Plaque stability may also be improved, decreasing the likelihood of plaque rupture and subsequent thrombosis and stroke or myocardial infarction.

What are needed in the art are methods and compositions that may inhibit angiogenesis in pathological conditions such as atherosclerosis.

### SUMMARY

According to one embodiment, disclosed is a method for preventing angiogenesis through inhibition of function of an endothelial cell. More specifically, the method can include contacting an endothelial cell with sparstolonin B or a derivative thereof, the sparstolonin B contacting the endothelial cell at a concentration of from about 0.0001 micromolar (μM) to about 100 μM.

According to another embodiment, a composition for preventing angiogenesis is disclosed. The composition can include sparstolonin B or a derivative thereof and a pharmaceutically acceptable carrier. The composition can include the sparstolonin B or a derivative thereof in an amount such that an endothelial cell that is contacted with the composition will be contacted with the sparstolonin B in an amount of from about 0.0001 μM to about 100 μM.

### BRIEF DESCRIPTION OF THE FIGURES

A full and enabling disclosure of the present invention, including the best mode thereof, directed to one of ordinary skill in the art, is set forth in the specification, which makes reference to the appended figure, in which:

FIG. 1 illustrates endothelial cell tube formation following contact of cells with SsnB including total tube length as a function of SsnB concentration in HUVECs (FIG. 1A), total tube length as a function of SsnB concentration in HCAECs (FIG. 1B), (*p<0.05 vs. vehicle control, Newman-Keuls test), a micrograph of tube formation in HUVECs in a control system (FIG. 1C) and in a system including 100 μM of SsnB (FIG. 1D).

FIG. 2 illustrates SsnB inhibition of HUVEC migration including the graphical results of migrated cells as a function of SsnB concentration (0.1 to 100 μM) (FIG. 2A), the migrated cells as a function of SsnB concentration (0.0001 to 0.1 μM) (FIG. 2B) (*p<0.05 vs. vehicle control, Newman-Keuls test) and representative micrographs demonstrating cell migration including vehicle control (FIG. 2C), negative control (FIG. 2D), and 100 μM SsnB (FIG. 2E).

FIG. 3 illustrates the SsnB inhibition of tube formation following contact with HMVECs.

FIG. 4 illustrates SsnB arrest of endothelial cells in the G1 phase of the cell cycle. As shown in FIG. 4A, after 24, 30, and 36 hours of treatment, 100 μM SsnB decreased the percentage of cells in the S phase and increased the percentage of cells in the G1 phase (*p<0.005 vs. corresponding vehicle control, Newman-Keuls test). FIG. 4B presents representative cell cycle data demonstrating the increase in G1 cell percentage and decrease in S phase cell percentage after 24 hours of SsnB treatment (left—vehicle control, right—SsnB)

FIG. 5 illustrates SsnB arrest of endothelial cells in the G2 phase of the cell cycle.

FIG. 6 illustrates the normalized blood vessel length in a control and in a test protocol using SsnB.

FIG. 7 illustrates the normalized vessel branch number in an in vivo testing protocol for a control and a test protocol using SsnB.

FIG. 8 presents images of blood vessel development for vessels developed in conjunction with SsnB (FIG. 8A) and a control (FIG. 8B).

### DETAILED DESCRIPTION OF THE INVENTION

The following description and other modifications and variations to the present invention may be practiced by those of ordinary skill in the art, without departing from the spirit and scope of the present invention. In addition, it should be understood that aspects of the various embodiments may be interchanged in whole and in part. Furthermore, those of ordinary skill in the art will appreciate that the following description is by way of example only, and is not intended to limit the invention.

In general, disclosed herein is a method for preventing angiogenesis and compositions that may be utilized in carrying out the method. More specifically, the disclosure is directed to the anti-angiogenic effects of sparstolonin B (SsnB). The disclosed methods and compositions can be utilized to encourage an anti-angiogenic effect through application of SsnB or a derivative thereof to endothelial cells. Without wishing to be bound to any particular theory, it is hypothesized that SsnB can inhibit angiogenesis by targeting endothelial cell cycle progression, cell migration, and chemotaxis. Moreover, SsnB can exhibit many diverse effects, including anti-inflammatory, anti-angiogenic, and cytostatic properties, by which SsnB may have potential therapeutic uses for a plethora of disorders.

Sparstolonin B (SsnB) is a bioactive compound that can be isolated from the plant Sparganium stoloniferum. Sparganium stoloniferum is a perennial, aquatic plant grown in North and East China that has long been used in Traditional Chinese Medicine (TCM) for the treatment of several inflammatory diseases and as an anti-spasmodic and anti-tumor agent. Current work with this herb has mainly dealt with its extracts. Extracts containing active components have been isolated from the root-like modified stem of the plant, known as the rhizome or tuber. A typical extraction protocol involves drying the rhizome and grounding it into a powder. The powder is mixed in boiling distilled water to produce an aqueous solution. Extracts may also be prepared with organic solvents, including ethanol, choloroform, and ethyl acetate. Extracts contain a multitude of active components including flavonoids, phenylpropanoid glycosides, aromatic acids, and polyphenols.

Previously discovered extracts and other isolated chemical compounds from this herb, including a sucrose ester, a phenylpropanoid glycerol, carboxylic acid esters, and a phenylpropanoid glycoside, have demonstrated anti-cancer effects.

The structure of SsnB is provided below.

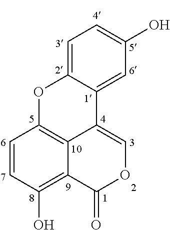 [SMILES: O=c1occ2c3c(ccc(O)c13)Oc1ccc(O)cc1-2]

SsnB and derivatives thereof are encompassed in the present disclosure. Derivatives can include modifications to the basic structure that do not affect the efficacy of the material. For example, in one embodiment the hydroxyl group at the 5′ and/or 8 carbon of the molecule can be replaced with a group to affect the solubility of the SsnB. By way of example, one or both of these hydroxyl groups can be replaced with a more hydrophilic group, such as a carboxyl group, a carbonyl group, a sulfhydryl group, a phosphate group, an amine group, combinations thereof, and so forth.

Prior research has shown that SsnB exhibits strong anti-inflammatory effects on mouse and human macrophages, selectively inhibiting the inflammatory responses of macrophages to ligands for Toll-like Receptors 2 and 4 (TLR2 and TLR4). SsnB has also been shown to suppress downstream signaling pathways after TLR2 and TLR4 activation, including MAPK and NF-κB. These findings suggest that SsnB may be an antagonist to TLR2 and TLR4.

The effects that SsnB may have on biological pathways may be revealed by its chemical structure. NMR and X-ray crystallography have identified SsnB as a polyphenol with structural similarities to isocoumarins and xanthone. Both of these compounds are known for anti-coagulant, anti-inflammatory, and anti-tumor properties. Polyphenolic compounds derived from plants, including olives and grapes, have been shown to have anti-oxidant, anti-inflammatory, cardioprotective, and anti-cancer properties. Compounds derived from green tea and red wine, particularly quercetin and resveratrol, have both demonstrated angiogenesis inhibition. While not wishing to be bound to any particular theory, it is believed that the ability of SsnB to serve as an angiogenesis inhibitor may be related to the polycyclic/polyphenolic structure of SsnB.

The presently disclosed methods and compositions take advantage of the routes by which SsnB can affect endothelial cells and thereby prevent angiogenesis. As an effective inhibitor of angiogenesis, SsnB can be a suitable therapeutic alternative for treatment of disorders in which excessive blood vessel growth contributes to the pathology, including cancer, rheumatoid arthritis, and psoriasis. SsnB may particularly be suited for treatment of atherosclerosis due to its combined anti-angiogenic and anti-inflammatory properties. Inhibiting angiogenesis may prevent intraplaque angiogenesis, thereby limiting plaque growth, destabilization, and subsequent rupture. At the same time, SsnB may inhibit the chronic inflammatory reactions necessary for plaque formation and growth.

Targeting endothelial cell proliferation has shown potential in the area of angiogenesis inhibition. Endothelial cell proliferation may be inhibited in numerous ways, including the downregulation of cell cycle regulatory proteins. Cyclins and cyclin dependent kinases are regulatory proteins that control progression through the cell cycle by regulating specific cell cycle checkpoints. Cyclins help activate cyclin dependent kinases, which phosphorylate downstream proteins that allow cells to progress through these checkpoints. Cyclin E2 (CCNE2) and Cell division cycle 6 (CDC6) are regulatory proteins that control progression through the G1/S checkpoint. CDC6 regulates DNA replication, and cyclin E2 activates cyclin-dependent kinase 2. Downregulation of CCNE2 and CDC6 will trap cells at the G1/S checkpoint and prevent endothelial cells from initiating DNA replication.

While not wishing to be bound to any particular theory, it is believed that SsnB can function as an anti-angiogenic factor through inhibition of the cell cycle progression. As described further herein, microarray experiments with human endothelial cells demonstrate that SsnB exposure can promote differential expression of several hundred genes (916 and 356 genes, respectively, with fold change ≧2, p<0.05, unpaired t-test). Microarray data from both cell types present significant overlap, including genes associated with cell proliferation and cell cycle. Flow cytometric cell cycle analysis of HUVECs treated with SsnB show an increase of cells in the G1 phase and a decrease of cells in the S phase. As discussed above, cyclin E2 (CCNE2) and Cell division cycle 6 (CDC6) are regulatory proteins that control cell cycle progression through the G1/S checkpoint. In one embodiment contact of an endothelial cell with SsnB can downregulate both CCNE2 and CDC6. Real Time quantitative PCR illustrate that CCNE2 and CDC6 in HUVECs can be downregulated after SsnB exposure. For instance, CCNE2 can be downregulated to a concentration of about 64% as compared to a control and CDC6 can be downregulated to a concentration of about 35% as compared to a control following contact of the endothelial cell with SsnB. The SsnB downregulation of CCNE2 and CDC6 through contact of an endothelial cell can halt progression of the cell cycle through the G1/S checkpoint.

During angiogenesis, the basement membrane of the blood vessel is broken down, and endothelial cells migrate into the surrounding tissue and proliferate. After migration and proliferation, the endothelial cells form a new lumen and start to secrete extracellular matrix molecules, ultimately forming a new capillary. While not wishing to be bound to any particular theory, it is believed that SsnB can inhibit endothelial cell migration and tube formation, additional steps required for angiogenesis, by modulating cytoskeletal reorganization.

At concentrations up to 100 μM, SsnB does not exhibit cytotoxic effects on various cell types, including mouse peritoneal macrophages, HUVECs, human aortic smooth muscle cells, and monocytic THP-1 cells. Accordingly, disclosed methods can include applying SsnB to an endothelial cell at a concentration at or less than about 100 μM.

With regard to specific effects of SsnB contact with endothelial cell(s), contact of a plurality of endothelial cells with SsnB at a concentration of from about 0.01 μM to about 100 μM, or from about 1 μM to about 100 μM, can inhibit tube formation by the cells. The effect of SsnB on cell migration can be even stronger, with SsnB inhibiting cell migration of an endothelial cell at a contact concentration of from about 0.0001 μM to about 100 μM, or from about 0.0001 μM to about 1 μM, or from about 0.0001 μM to about 0.1 μM.

The disclosed methods and compositions can be utilized for both in vitro (e.g., in laboratory testing) applications and in vivo (e.g., treatment) applications. For example, tissues that incorporate endothelial cells can exhibit significant reduction in capillary length and branching relative to a vehicle control group. SsnB can cause a significant reduction in in vivo angiogenesis.

In one embodiment, SsnB can be provided as a biocompatible composition. A composition can generally include the SsnB in a concentration that can vary over a wide range, with a preferred concentration generally depending on the particular application, the delivery site and the mode that will be used in the delivery process. For example, a composition can include SsnB at a concentration of from about 0.0001 μM to about 0.5 M, or from about 0.0001 μM to about 0.1 M so as to contact an endothelial cell at a concentration of between about 0.001 μM and about 100 μM. It should be noted, however, that while these exemplary concentrations are effective in certain embodiments, the composition can include a wider range of SsnB concentrations. For example, actual concentrations used may be influenced by the tissue targeted by the procedure, size of the targeted area, desired incubation time, and preferred pH, in addition to delivery mode.

In one embodiment, SsnB can be provided in pharmaceutically acceptable formulations using formulation methods known to those of ordinary skill in the art. These formulations can generally be targeted to endothelial cells by standard routes. For example, the formulations may be administered in one embodiment directly to endothelium, for instance through exposure of the endothelium and direct application thereto, or via direct injection of the formulation to the targeted endothelium. In other embodiments, however, the formulations may be administered indirectly to the targeted tissue.

The composition can be delivered intravenously in a systemic delivery protocol. For example, osmotic mini-pumps may be used to provide controlled delivery of high concentrations of the treatment agents through cannulae to the site of interest, such as directly into a targeted blood vessel.

A composition can include additional agents, in addition to the SsnB. Such agents can be active agents, providing direct benefit to the tissue, or may be supporting agents, improving delivery, compatibility, or reactivity of other agents in the composition.

A composition can include one or more buffers as are generally known in the art. For example, a composition including SsnB and having a pH from about 4.0 to about 9.0 may be formulated with inclusion of a biocompatible buffer such as distilled water, saline, phosphate buffers, borate buffers, HEPES, PIPES, and MOPSO. In one embodiment, a composition may be formulated to have a pH of between about 5.5 and about 7.4.

Compositions for parenteral delivery, e.g., via injection, can include pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (e.g., glycerol, propylene glycol, polyethylene glycol and the like), carboxymethylcellulose and suitable mixtures thereof, vegetable oils (e.g., olive oil) and injectable organic esters such as ethyl oleate. In addition, the composition can contain minor amounts of auxiliary substances such as wetting or emulsifying agents, pH buffering agents and the like that can enhance the effectiveness of the phenolic compound. Proper fluidity may be maintained, for example, by the use of coating materials such as lecithin, by the maintenance of the required particle size in the case of dispersions and by the use of surfactants. These compositions may also contain adjuvants such as preservatives, wetting agents, emulsifying agents and dispersing agents.

Prevention of the action of microorganisms may be ensured by the inclusion of various antibacterial and antifungal agents such as paraben, chlorobutanol, phenol, sorbic acid and the like. It may also be desirable to include isotonic agents such as sugars, sodium chloride and the like.

In one embodiment, the compositions can include pharmaceutically acceptable salts of the components therein, e.g., those that may be derived from inorganic or organic acids. Pharmaceutically acceptable salts include the acid addition salts that are formed with inorganic acids such as, for example, hydrochloric or phosphoric acids, or such organic acids as acetic, tartaric, mandelic and the like. Salts formed with free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptonoate, hexanoate, fumarate, hydrochloride, hydrobromide, hydroiodide, 2-hydroxymethanesulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl, and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

In one embodiment the method can include use of timed release or sustained release delivery systems as are generally known in the art. Such systems can be desirable, for instance, in situations where long term delivery of the agents to a particular location is desired. According to this particular embodiment, a sustained-release matrix can include a matrix made of materials, usually polymers, which are degradable by enzymatic or acid/base hydrolysis or by dissolution. Once located at or near the target tissue, e.g., inserted into the body, for instance in the form of a patch or a stent, such a matrix can be acted upon by enzymes and body fluids. The sustained-release matrix can be chosen from biocompatible materials such as liposomes, polylactides (polylactic acid), polyglycolide (polymer of glycolic acid), polylactide co-glycolide (co-polymers of lactic acid and glycolic acid) polyanhydrides, poly(ortho)esters, polyproteins, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone.

In one embodiment, the SsnB can be targeted by use of a hydrogel delivery vehicle. Hydrogels include polymeric matrices that can be highly hydrated while maintaining structural stability. Suitable hydrogel matrices can include un-crosslinked and crosslinked hydrogels. In addition, crosslinked hydrogel delivery vehicles of the invention can optionally include hydrolyzable portions, such that the matrix can be degradable when utilized in an aqueous environment, e.g., in vivo. For example, the delivery vehicle can include a crosslinked hydrogel including a hydrolyzable cross-linking agent, such as polylactic acid, and can be degradable in vivo.

Hydrogel delivery vehicles can include natural polymers such as glycosaminoglycans, polysaccharides, proteins, and the like, as well as synthetic polymers, as are generally known in the art. A non-limiting list of hydrophilic polymeric materials that can be utilized in forming hydrogels can include dextran, hyaluronic acid, chitin, heparin, collagen, elastin, keratin, albumin, polymers and copolymers of lactic acid, glycolic acid, carboxymethyl cellulose, polyacrylates, polymethacrylates, epoxides, silicones, polyols such as polypropylene glycol, polyvinyl alcohol and polyethylene glycol and their derivatives, alginates such as sodium alginate or crosslinked alginate gum, polycaprolactone, polyanhydride, pectin, gelatin, crosslinked proteins peptides and polysaccharides, and the like.

Delivery vehicles can also include vascular grafts. For example, an allograft, xenograft or autologous graft can be associated with SsnB prior to implantation. For example, the vascular graft can be coated with SsnB or a composition including SsnB as herein described. During implantation, the vascular graft can be located in association with the targeted tissue, and thus serve to deliver the SsnB to the tissue.

A delivery system can include a combination of one or more delivery vehicles. For instance, a hydrogel delivery vehicle can be combined with a patch, a stent, a perforated balloon, a vascular graft, or any other suitable device, for delivery of the SsnB to targeted endothelium.

Disclosed methods to inhibit angiogenesis may hold the key to new treatments for not only atherosclerosis, but also various other angiogenesis-related pathological conditions, including psoriasis, cancer, and rheumatoid arthritis.

The disclosure may be better understood with reference to the following examples.

### Example 1

### Experimental Procedures

### Materials

Sparstolonin B was purified from the plant Sparganium stoloniferum according to standard methods as are known in the art. Briefly, sparstolonin B isolation was begun by soaking the powdered tuber in 85% ethanol overnight and extracting three times with the same solvent. The solvent was filtered and concentrated under vacuum, producing a residue, which was then dissolved in water and extracted with petrol, ethyl acetate, and n-butyl alcohol in sequential order. This extract was run through a silica gel column and eluted with petrol-ethyl acetate mixtures with increasing polarities. The end product was yellow needles containing SsnB. The purity of the SsnB was determined to be greater than 99% by HPLC, and a stability test was utilized to ensure that samples were consistently >99% pure.

### Cell Culture

Human coronary artery endothelial cells (HCAECs), human umbilical vein endothelial cells (HUVECs), and human cardiac microvascular endothelial cells (HMVECs) were obtained from Lonza (Hopkinton, Mass.) and cultured on polystyrene, tissue culture-treated petri plates (100×20 mm) coated with 0.1% gelatin. HUVECs, HCAECs, and HMVECs were cultured in endothelial cell medium supplemented with 10% fetal bovine serum (FBS) and endothelial cell mitogen/growth supplement (Biomedical Technologies, Stoughton, Mass.). The endothelial cell medium was replaced every 2-3 days, and the cells were passaged after complete confluence was reached. Confluent plates were trypsinized and split, and the cells were cultured until the fourth passage was reached.

### Matrigel Tube Formation Assay

To initially determine if SsnB inhibited pro-angiogenic cell functions, a tube formation assay with Matrigel was performed. Growth factor reduced Matrigel (BD Biosciences, Bedford, Mass.) was added to the wells of a 96 well polystyrene culture plate and incubated at 37° C. for 30 minutes. Cells (HUVECs, HCAECs, or HMVECs at passage 2 to 4) were added to each well to reach a final number of 20,000 cells per well. SsnB was added to the wells at a concentration of 1, 10, or 100 μM. Endothelial cell medium with DMSO (0.1%) was used as a vehicle control. Each group contained 4 replicates. The plates were placed in an incubator for 4 h. During the incubation, the endothelial cells formed elongated structures called cords, also known as tubes. After 4 h, neutral buffered formalin was added to fix the cells. Pictures of three non-overlapping fields were taken from each well. The lengths of single cell endothelial cords were measured with Image-Pro Plus (Media Cybernetics, Silver Spring, Md.), and the sum of tube lengths for each well was determined. The average total length and standard deviation for each group were determined, and the appropriate statistical tests (ANOVA and Newman-Keuls) were completed. The tube formation assay was replicated three times for both HUVECs and HCAECs. The assay was also repeated with cardiac HMVECs.

### Cell Viability

A Live/Dead assay (Invitrogen, Eugene, Oreg.) was utilized to investigate the effect of SsnB on cell viability. The Matrigel tube formation experiment was repeated with HUVECs in chamber slides at a concentration of 20,000 cells per well. The cells were treated with SsnB (1, 10, or 100 μM) or Vehicle Control (0.1% DMSO) as described above. After four hours of incubation, the slides were removed. A chamber slide containing HUVECs treated with 70% methanol for 30 minutes was used as a control for dead cell staining. The slides were aspirated and washed with PBS, and EthD-1 and calcein AM were added to each well. The plates were incubated in the dark, and images were taken with a light microscope at 10× magnification.

### Transwell Insert Cell Migration Assay

The cell invasion assay was performed with a Transwell insert system (6.5 mm diameter inserts with 8.0 μm pores in a polycarbonate membrane situated in wells of 24 well polystrene, tissue culture treated plates, Corning Incorporated, Corning, N.Y.). The Transwell inserts were coated with 0.1% gelatin for 30 min and incubated in low serum medium for 1 h. Cultured HUVECs were trypsinized and then resuspended in low serum medium (0.5% fetal bovine serum without endothelial cell mitogen), and added at 50,000 cells per insert. The cells were allowed to adhere to the inserts for 30 min. Next, various concentrations of SsnB (0.0001, 0.001, 0.01, 0.1, 1, 10, and 100 μM) or vehicle control (0.1% DMSO) were added to the Transwell inserts. After 30 min, the medium in the lower chamber for the experimental groups was replaced with low serum medium containing 10 ng/ml VEGF and thus establishing a chemoattractant gradient between the top insert and lower chamber. For the negative control group, the medium was replaced with low serum medium (0.5% fetal bovine serum). The plates were incubated for 8 h at 37° C. During the incubation, the cells migrated through the pores of the Transwell insert towards the lower chamber. At the end of this period, cells on the upper surface of the insert were removed, and migrated cells on the bottom side were fixed in formalin and stained with Hoechst dye (a fluorescent nuclear stain). The filter inserts were removed from the wells and mounted on glass slides. Cells were counted from four random fields observed with a 10× objective lens. The cell migration experiments were repeated three times at high SsnB concentrations (0.1, 1, 10, 100 μM) and one time at low SsnB concentrations (0.0001, 0.001, 0.01, 0.1 μM) for HUVECs.

### Cell Cycle Analysis

Cell cycle analysis was performed using propidium iodide staining and flow cytometry. HUVECs (approximately 75% confluent) cultured in 6 well polystyrene culture plates coated with 0.1% gelatin were serum starved for 24 hours in low serum medium containing 0.5% fetal bovine serum and no endothelial cell mitogen to synchronize the cells in the G1/S phase. After 24 hours, the low serum medium was replaced with treatments of either 100 μM SsnB or vehicle control (0.1% DMSO) diluted in complete growth medium (10% fetal bovine serum with endothelial cell mitogen) in triplicate. The treated cells were incubated for 24, 30, and 36 hours. After incubation, the cells were trypsinized, transferred to 5 ml polysytrene round bottom tubes (12×75 mm), and centrifuged. The medium was aspirated, and the cells were washed with PBS. After fixing the cells with ice-cold 70% ethanol for 15 min, the cells were centrifuged and stained with propidium iodide for 30 min. The samples were then analyzed with a flow cytometer (Beckman Coulter FC500). The unstained and stained cell groups were used to calibrate the settings on the flow cytometer. The data were collected and analyzed with ModFit software.

### Microarray Analysis

Confluent plates (100×20 mm polystyrene, tissue culture-treated petri plates coated with 0.1% gelatin, 75% confluent) of HUVECs and HCAECs (four plates for HUVECs, n=2, and six plates for HCAECs, n=3) were chosen for the microarray experiments. Half of the plates received complete growth medium (10% fetal bovine serum with endothelial mitogen) containing vehicle control (0.1% DMSO), and the remaining plates received complete growth medium containing 100 μM SsnB. The plates were incubated for 24 h to allow SsnB to have an effect on cellular gene expression. Following incubation, RNA isolation was completed with the RNeasy Mini kit from Qiagen. The cells were lysed, and RNA was isolated by using the RNeasy spin columns and following the protocol provided by Qiagen. Purified RNA was sent to the Medical University of South Carolina Proteogenomics Facility for microarray analysis. The GeneChip Human Genome U133 Plus 2.0 Array was utilized to track changes in gene expression due to SsnB treatment. Complete data was uploaded to the NCBI Gene Expression Omnibus database (accession number GSE44598).

### Real Time RT-PCR

After a careful analysis of the microarray data, key genes (CDC6, CCNE2, KITLG, ALDH3A1, CCNB1, CDC2, HMMR, DIAPH3, ANLN, and CDKN3) were chosen for quantitative real-time PCR (qRT-PCR) to verify the gene expression results. HCAECs and HUVECs were exposed to SsnB or vehicle control for 24 h (as previously described in the microarray section). For RT-PCR, RNA was isolated from the cells with the RNeasy Mini kit as described previously. After forward and reverse primer kits (Qiagen, Valencia, Calif.) were selected, the RNA was amplified. The one-step RT-PCR reactions were completed on the BioRad iCycler thermal cycler system in the Instrumentation Resource Facility at the USC School of Medicine. The expression levels were normalized, and RNA levels were quantified.

### Data Analysis

Microarray data analysis, including data normalization (robust multi-array average), identification of differentially expressed genes (comparative analyses with dChip software), and heat map construction, was carried out to determine how SsnB affects gene expression and affected pathways (n=2 for HUVECs and n=3 for HCAECs). After a careful analysis of the microarray data, several key genes were chosen for qRT-PCR to verify the gene expression results. The genes were chosen based on the following criteria: fold change ≧2, p<0.05, unpaired t-test with a false discovery rate approximating 0%, appearing in both data sets (HUVECs and HCAECs), and gene function relating to cell proliferation and/or angiogenesis. For qRT-PCR, the expression levels were normalized to the housekeeping gene GAPDH, and RNA levels were compared between groups with the ΔΔCₜ method.

### Results

### SsnB Inhibits Endothelial Cell Tube Formation and Cell Migration

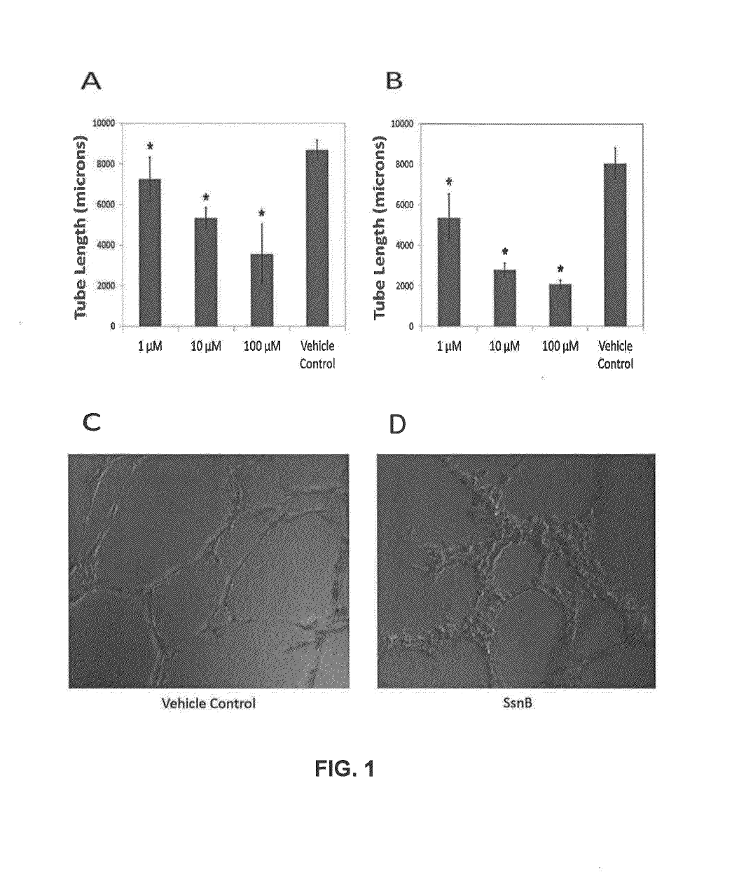

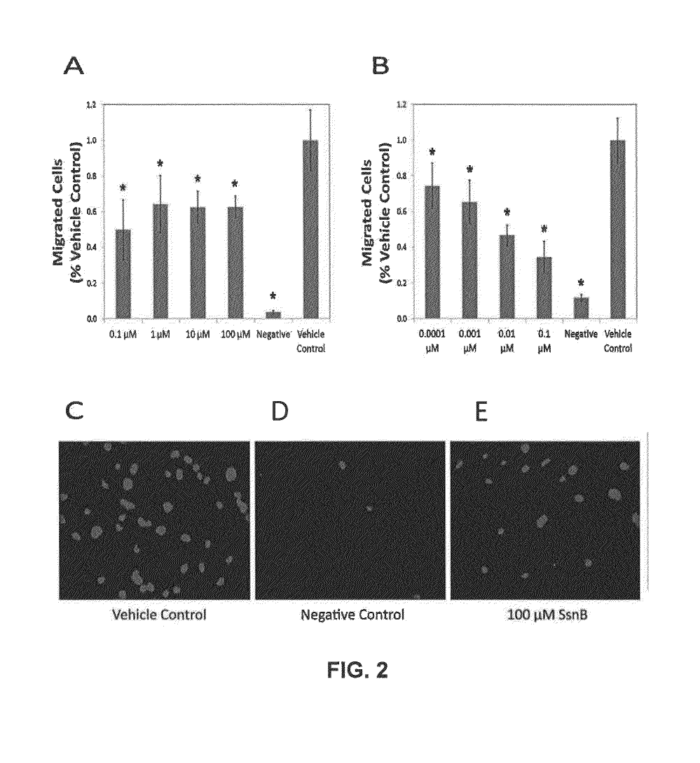

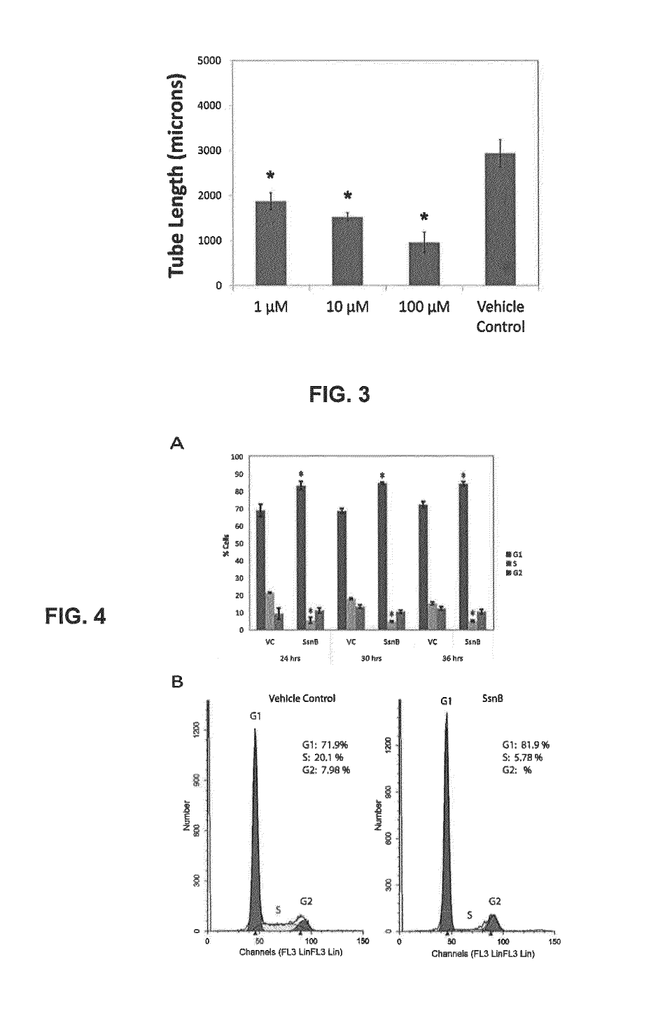

FIG. 1 an FIG. 2 show representative results from the tube formation and cell migration assays. As can be seen, SsnB treatment resulted in a dose-dependent inhibition of HUVEC tube formation at concentrations between 1 and 100 μM (p<0.05, Newman-Keuls test). SsnB also demonstrated a dose-dependent inhibition of VEGF-induced cell migration at concentrations between 0.0001 and 0.1 μM (p<0.05), which leveled off between 0.1 and 100 μM (p<0.05). Experiments with HCAECs also demonstrated a dose-dependent inhibition (between 1 and 100 μM) of endothelial cell tube formation on the substrate Matrigel (FIG. 1B). A dose-dependent inhibition of tube formation was also seen with cardiac HMVECs (FIG. 3). Results from the live/dead assay with HUVECs showed that SsnB had no effect on cell viability at the concentrations used. Cells treated with SsnB or vehicle control showed positive staining for live cells, but no staining for dead cells. These data suggest that SsnB is able to inhibit endothelial cell morphogenesis and that cell migration, a crucial process in angiogenesis, may play a role.

### SsnB Arrests Endothelial Cells in the G1 Phase of the Cell Cycle

FIG. 4 depicts representative results from the cell cycle experiments. In comparison to vehicle controls, flow cytometric cell cycle analysis of HUVECs treated with SsnB showed an increase of cells in the G1 phase and a decrease of cells in the S phase after 24, 30, and 36 hours of treatment. In untreated cells, the percentage of cells in S phase decreased and the percentage in G2/M increased from 24 to 36 hours after addition of growth medium, as expected once cells begin to re-enter and move through the cell cycle. Overall, these results imply that endothelial cells are being arrested in the G1/S phase, suggesting an inhibition of cell proliferation, an important step in angiogenesis.

SsnB Changes the Expression of Genes Associated with Cell Cycle and Cell Proliferation

Microarray experiments demonstrated differential expression of several hundred genes in response to SsnB exposure (916 genes for HUVECs and 356 genes for HCAECs, fold change ≧2, p<0.05, unpaired t-test with a false discovery rate approximating 0%). Overall, microarray data from both cell types showed significant overlap, including genes in pathways associated with cell proliferation, cytoskeleton, chemotaxis, and cell cycle, all areas implicated in angiogenesis. These results are consistent with the data obtained from the cell migration and cell cycle functional studies. From this microarray study, it is clear that SsnB regulates genes involved in angiogenic processes in HUVECs and HCAECs.

### Real Time RT-PCR

Following microarray data analysis, certain genes (listed in Table 1) were chosen for verification with real time RT-PCR. Cyclin E2 (CCNE2) and Cell division cycle 6 (CDC6) are regulatory proteins that control cell cycle progression through the G1/S checkpoint. Both CCNE2 and CDC6 were downregulated in the microarray data. qRT-PCR confirmed that gene expression of CCNE2 and CDC6 was downregulated after SsnB exposure to 64% and 35% of controls respectively for HUVECs and to 57% and 14% of controls respectively for HCAECs. Kit-Ligand (KITLG), also known as stem cell factor, is a protein involved in the differentiation and growth of stem cells. Aldehyde dehydrogenase 3 family, member A1 (ALDH3A1) is a protein involved in the aryl hydrocarbon receptor pathway. These genes were chosen for further study because they were highly upregulated by SsnB treatment in the microarray data set. In HUVECs, qRT-PCR analysis demonstrated that KITLG and ALDH3A1 expression was upregulated to 400% and 1280% of controls, respectively. In HCAECs, KITLG and ALDH3A1 expression was upregulated to 260% and 4620% of controls, respectively. The microarray data was supported in both data sets.

TABLE 1

| | HUVECs | | | HCAECs | | |
|---|---|---|---|---|---|---|
| | | Microarray | | | Microarray | |
| | PCR | Fold | | PCR | Fold | |
| | 2^{ΔΔCt} | change | | 2^{ΔΔCt} | change | |
| Gene | value | value | p-value | value | value | p-value |
| KITLG | 4.0 | 6.2 | 0.02795 | 2.6 | 2.1 | 0.00010 |
| ALDH3A1 | 12.8 | 7.7 | 0.00609 | 46.2 | 8.8 | 0.00001 |
| HMMR | 0.24 | 0.11 | 0.03073 | 0.062 | 0.20 | 0.00006 |
| DIAPH3 | 0.19 | 0.34 | 0.03248 | 0.26 | 0.38 | 0.00019 |
| ANLN | 0.20 | 0.09 | 0.00900 | 0.13 | 0.11 | 0.000001 |
| CDKN3 | 0.18 | 0.19 | 0.01471 | 0.22 | 0.18 | 0.00018 |
| CCNB1 | 0.40 | 0.19 | 0.02304 | 0.13 | 0.16 | 0.00003 |
| CDC2 | 0.14 | 0.08 | 0.00440 | 0.17 | 0.20 | 0.00004 |
| CDC6 | 0.35 | 0.23 | 0.01527 | 0.14 | 0.18 | 0.00185 |
| CCNE2 | 0.64 | 0.24 | 0.01814 | 0.57 | 0.38 | 0.00180 |

Data showed that SsnB affected the gene expression of additional cell cycle regulatory proteins, including cyclin B1 (CCNB1) and cyclin dependent kinase 1 (CDC2). CCNB1 and CDC2 are regulatory proteins that control progression through the G2/M checkpoint. Downregulation of CCNB1 and CDC2 will trap cells at the G2/M checkpoint and prevent endothelial cells from entering the final stages of cell division. Table 1 demonstrates that CCNB1 and CDC2 were both downregulated by SsnB.

The microarray data for both HUVECs and HCAECs demonstrated an enrichment of genes associated with both pathways, including diaphanous homolog 3 (DIAPH3), hyaluronan-mediated motility receptor (HMMR), and anillin (ANLN), an actin binding protein.

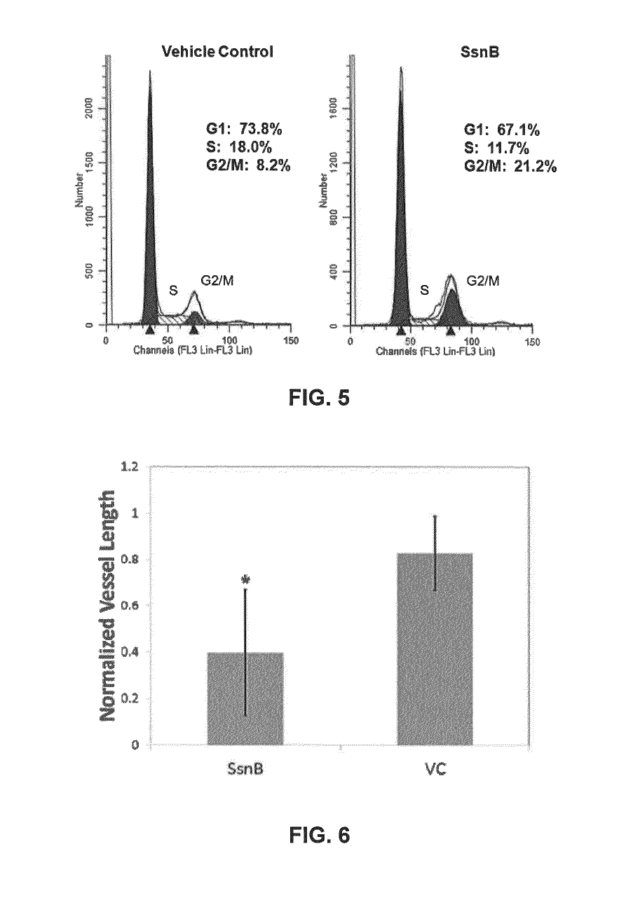

Table 1 demonstrates that CCNE2, CDC6, CCNB1, and CDC2 were all downregulated by SsnB. The downregulation of CCNE2 and CDC6 directly supports the G1/S blockage observed in the cell cycle experiments with synchronized endothelial cells. The microarray data also showed a downregulation of CCNB1 and CDC2, which should trap cells at the G2/M checkpoint and prevent endothelial cells from entering the final stages of cell division. In non-synchronized (no serum starvation) HUVECs, G2/M blockage was observed (FIG. 5) but at much lower levels compared to the G1/S blockage.

In addition to the cell cycle regulatory genes, there were additional genes associated with mitosis and the cytoskeleton that stood out in the microarray data. The microarray data showed enrichment in genes encoding proteins associated with the spindle, nuclear envelope, chromosome condensation and segregation, and cytokinesis, all important aspects of mitosis and cell division. SsnB caused a downregulation in the expression of anillin (ANLN), an actin binding protein that plays an important role in cytokinesis, the final stage of cell division. This could inhibit contractile ring formation, preventing the cytoskeletal changes necessary for cytokinesis. Decreased expression of anillin might explain the nuclei clumping or double nuclei that we observed in additional experiments with nuclear staining (Hoechst). The microarray data also contained many examples of differentially downregulated cytoskeleton-related genes, including transforming acidic coiled-coil containing protein 3 (TACC3), hyaluronan-mediated motility receptor (HMMR), and diaphanous homolog 3 (DIAPH3). Downregulation of the protein products of these genes may have a negative influence on cytoskeleton functioning, which is essential for not only mitosis and cytokinesis, but for cell motility as well. DIAPH3 is an essential element in filopodia and lamellopodia formation, both important processes for cell motility. Downregulation of DIAPH3 may inhibit lamellopodialfilopodia protrusion and subsequent cell spreading. This may explain the cell rounding that we noticed in the tube formation experiments. TACC3 mainly controls microtubules and is intricately involved with the centrosome and mitotic spindle apparatus during mitosis. The spindle apparatus is required for separation of sister chromatids and subsequent nuclear division. Any disruption of this process could negatively affect cell proliferation. Furthermore, aurora kinases (AURKA and AURKB), which were also downregulated in the data, help regulate TACC3-mediated formation of the mitotic spindle apparatus. Hyaluronan-mediated motility receptor (HMMR or RHAMM) is a protein that regulates microtubule function during mitosis and the turnover of focal adhesions. RHAMM has been shown to be important for endothelial cell migration during angiogenesis. Antibodies directed against RHAMM effectively inhibited angiogenesis associated with cancer. RHAMM was also downregulated in the microarray data, suggesting its involvement in the inhibition of endothelial cell migration and angiogenesis.

Kinesin family member 18A (KIF18A) is another gene that was downregulated in the microarray data. Its functions encompass a variety of pathways, including the cytoskeleton, cell cycle progression, and cell division, which are all the main mechanistic processes that we believe SsnB targets to inhibit angiogenesis. KIF18A is a molecular motor that produces force and movement along microtubules to aid in cytoskeleton re-arrangement, especially chromosome movement. KIF18A has also been shown to interact with caveolin-1 (CAV1). Caveolin-1 is involved in cellular pathways associated with cell morphology and stress fiber formation. Silencing of caveolin-1 has been shown to inhibit angiogenesis through multiple mechanisms including alterations in cell morphology (increased cell size and stress fiber formation) and inhibition in cell migration and progression through the G1/S checkpoint. These findings connect especially well with present results that demonstrated a similar behavior in endothelial cells in response to SsnB treatment. It is hypothesize that SsnB may inhibit caveolin-1-mediated activities through downregulation of KIF18A. Caveolin-1 also signals through the AKT pathway; SsnB may somehow interfere with this signaling pathway.

### Example 2

To investigate the effects of SsnB on in vivo angiogenesis, a chick chorioallantoic membrane (CAM) assay was utilized. Chick embryos were exposed to methylcelluose discs containing vehicle control (DMSO) or 100 micromolar SsnB. Chick embryos receiving SsnB discs showed significant reduction in capillary length and branching number relative to the vehicle control group. Overall, SsnB caused a significant reduction in angiogenesis, demonstrating its in vivo efficacy.

### Experimental Procedures

### Materials

Sparstolonin B was purified from the plant Sparganium stoloniferum according to methods described above. Fresh fertilized bovans eggs were obtained from Clemson University and stored at 4° C. for no longer than one week.

### Methylcellulose Discs

Methylcellulose discs were created with a solution of 0.5% methylcellulose (diluted in sterile deionized water). A methylcellulose solution of 100 micromolar SsnB was made by adding 1.1 microliters of stock SsnB (93 mM) to 1 ml 0.5% methylcellulose. A vehicle control solution was created by adding 1 microliter of dimethyl sulfoxide (DMSO) to 1 ml 0.5% methylcellulose. Discs were created by placing 20 microliter droplets of SsnB or vehicle control solution onto polytetrafluoroethylene tape (to minimize sticking during drying) and allowed to dry under a laminar flow hood.

### Chick Chorioallantoic Membrane Assay

The fertilized eggs were placed in a humidified egg incubator with forced air circulation at 37° C. The eggs were automatically rotated every three hours. After 4 days of incubation, the eggs were removed and cracked. Chick embryos with intact yolks were placed in 100 mm petri plates. Each petri plate was placed in a weigh boat containing 13 ml Moscona's buffer and covered with cellophane wrap with two holes for air circulation. The plates were placed in a water-jacketed incubator (37 C, 5% CO₂) for 2 days. After 2 days of incubation, the methylcellulose discs containing either SsnB or vehicle control were placed on top of the CAM of each embryo in the right upper quadrant in an area near the allantoic vessels. After 2 additional days of incubation, the CAMs were examined and photographed with a dissecting stereomicroscope. Images were taken of areas containing the discs and non-treated regions. Each group contained seven embryos (calculated with PS 3.0.5 software, effect size of 50%, standard deviation of 30%, statistical power of 0.8, and α=0.05). The CAM assay was completed two times.

### Image Analysis

Images were analyzed with ImagePro Plus software. Blood vessel length and branching number were utilized to measure the effect of SsnB on angiogenesis. The vessel length was measured for areas containing the discs and non-treated regions. Normalized vessel length was calculated by dividing the length values of the treated regions by those of the non-treated regions. The same process was repeated for measuring the branching number.

### Statistical Analysis

To determine if a statistically significant difference between the treatment groups in each experiment existed, a one-way analysis of variance test (ANOVA) was completed.

### Results

### SsnB Inhibits Angiogenesis in the CAM Assay

The results from the CAM assay are depicted in FIG. 6. Overall, SsnB resulted in a significant decrease in normalized blood vessel length compared to the vehicle control group (ANOVA, p<0.05).

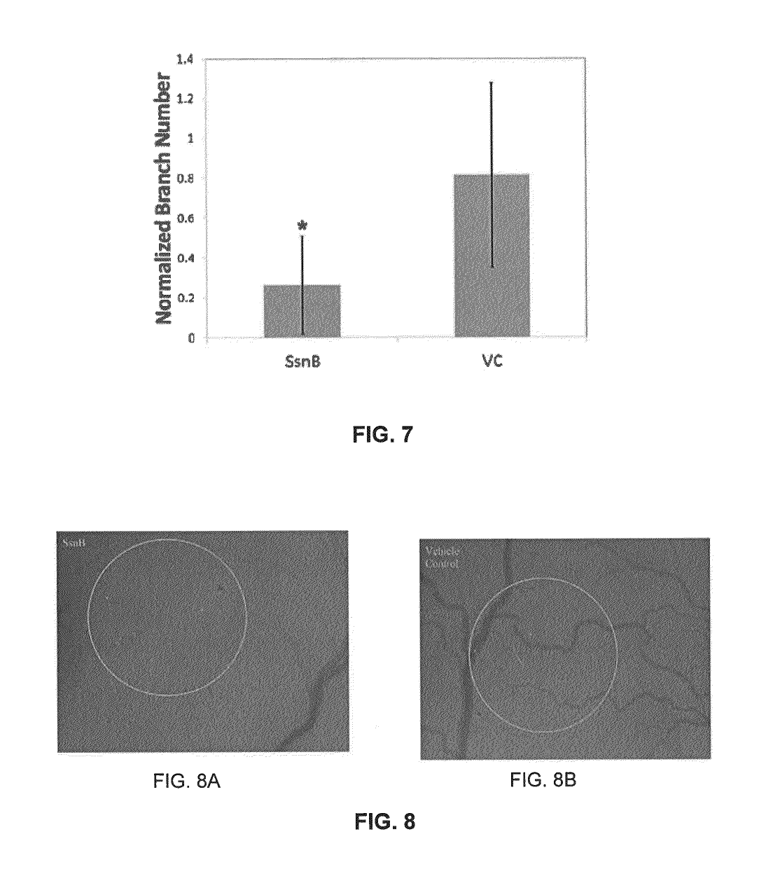

In addition to inhibiting blood vessel length, SsnB also caused a significant decrease in the normalized branch number. The results are depicted in FIG. 7. Compared to the vehicle control, SsnB decreased the number of points where blood vessels branched into two or more separate vessels. Interestingly, it has been shown that macrophage production of matrix metalloproteinases is important for blood vessel branching. SsnB may be inhibiting this process by blocking TLR activation of macrophages.

Representative images of the CAM from the SsnB and vehicle control groups after two days of treatment are shown in FIG. 8. The methylcellulose discs released SsnB or vehicle control and were still present at the end of the experiment, as complete biodegradation did not occur. The transparency of the methylcellulose discs allowed easy imaging of the vascular network underneath the discs. Images were taken of regions of the CAM that received methylcellulose discs as well as regions that did not receive discs. For each embryo, the blood vessel length and branch number were measured for both treated and non-treated areas. A normalized value was calculated by dividing the treated areas by the non-treated areas. This approach allowed for compensation for natural variations in vascular density among embryos that normally occur without the presence of angiogenic inhibitors or stimulators. Overall, the SsnB-treated group (FIG. 8A) showed fewer blood vessels, smaller vessel lengths, and fewer branches. The methylcellulose discs with SsnB not only inhibited angiogenesis in the area underneath the disc, but also demonstrated inhibition in the immediate area surrounding the discs. Compared to the SsnB group, the vehicle control group (FIG. 8B) exhibited higher vessel lengths, more vessels, and more vascular branching. Furthermore, SsnB treatment was associated with a decrease in blood vessel diameter and size.

In addition, SsnB had no effect on the viability or morphology of the embryos relative to the vehicle control. Both groups experienced similar number of embryo deaths, which is typically encountered with the CAM assay. The embryos that survived until the end of the experiment exhibited no obvious differences in size or morphology between the two groups.

Using the CAM assay, it has been demonstrated that SsnB effectively inhibits angiogenesis by decreasing total blood vessel length and by reducing branch number. This data demonstrates that SsnB is capable of inhibiting true, physiological angiogenesis.

Interestingly, SsnB caused a significant reduction in the number of branches in the developing CAM blood vessels. Blood vessel branch points have been shown to co-localize with macrophages that secrete matrix metalloproteinases (MMP) that are necessary for establishing these branch points. MMPs, especially MMP-9, help degrade the extracellular matrix, providing access routes for endothelial cells to migrate through the ECM to create new branches from the original blood vessels as angiogenesis progresses. The observed decrease in CAM vessel branching may reflect an inhibitory action on macrophages by SsnB. Previous research has shown that SsnB is capable of inhibiting macrophage-mediated inflammation by blocking toll-like receptor signaling. In addition to its effects on endothelial cells, SsnB may also be inhibiting macrophage production of MMPs or other proteolytic enzymes, thereby reducing the density of branching usually seen in angiogenesis in the CAM assay.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A method for preventing angiogenesis comprising applying a composition to an endothelial cell, the compositions comprising sparstolonin B or a derivative thereof in an amount such that the sparstolonin B contacts the cell at a concentration of from about 0.0001 micromolar to about 100 micromolar.

2. The method of claim 1, wherein the sparstolonin B or derivative thereof contacts the cell at a concentration of from about 0.0001 micromolar to about 0.1 micromolar, the method comprising inhibiting migration of the endothelial cell.

3. The method of claim 1, wherein the method comprises inhibiting tube formation by a plurality of endothelial cells.

4. The method of claim 1, the method comprising inhibition of the cell through the G1/S checkpoint of the cell cycle.

5. The method of claim 1, the method comprising inhibition of the cell through the G2/M checkpoint of the cell cycle.

6. The method of claim 1, wherein the method is carried out in vitro.

7. The method of claim 1, wherein the method is carried out in vivo.

8. The method of claim 7, wherein the cell is contacted with the composition via exposure of endothelium that comprises the cell and direct application of the composition to the cell.

9. The method of claim 7, wherein the cell is contacted with the composition via direct injection of the composition to endothelium that comprises the cell.

10. The method of claim 1, wherein the cell is a human endothelial cell.

11. The method of claim 1, wherein the cell is a coronary artery endothelial cell, an umbilical vein endothelial cell, or a cardiac microvascular endothelial cell.

12. The method of claim 1, wherein the composition comprises a derivative of sparstolonin B, the derivative including the substitution of a hydroxyl group at the 5′ and/or the 8 carbon of sparstolonin B with a carboxyl group, a carbonyl group, a sulfhydryl group, a phosphate group, an amine group, or combinations thereof.

13. A composition comprising sparstolonin B or a derivative thereof and a pharmaceutically acceptable carrier.

14. The composition of claim 13, the composition comprising the sparstolonin B or derivative thereof in an amount such that an endothelial cell that is contacted with the composition will be contacted with the sparstolonin B in an amount of from about 0.0001 micromolar to about 100 micromolar.

15. The composition of claim 13, wherein the composition comprises the sparstolonin B or derivative thereof in an amount of between about 0.0001 micromolar and about 10 molar.

16. The composition of claim 13, wherein the composition further comprises a buffer.

17. The composition of claim 13, wherein the composition further comprises an antibacterial and/or antifungal agent.

18. The composition of claim 13, wherein the composition is incorporated with a delivery vehicle.

19. The composition of claim 18, wherein the delivery vehicle comprises a hydrogel.

20. The composition of claim 18, wherein the delivery vehicle comprises a vascular graft.

21. The composition of claim 13, the composition comprising a derivative of sparstolonin B, the derivative including the substitution of a hydroxyl group at the 5′ and/or the 8 carbon of sparstolonin B with a carboxyl group, a carbonyl group, a sulfhydryl group, a phosphate group, an amine group, or combinations thereof.
